(19) United States
(12) Patent Application Publication
(10) Pub. No.: US 2024/0083828 A1
(43) Pub. Date: Mar. 14, 2024

(54) SYSTEM AND METHOD FOR PRODUCING VINYL CHLORIDE

(71) Applicant: FORMOSA PLASTICS CORPORATION, Yunlin County (TW)

(72) Inventors: Wen-Hsi HUANG, Kaohsiung City (TW); Sheng-Yen KO, Kaohsiung City (TW); Shih-Hong CHEN, Kaohsiung City (TW); Chun-Yu LIN, Kaohsiung City (TW)

(21) Appl. No.: 18/343,062

(22) Filed: Jun. 28, 2023

(30) Foreign Application Priority Data
Sep. 14, 2022 (TW) 111134647

(51) Int. Cl. C07C 17/25; B01J 6/00; B01J 19/00

(52) U.S. Cl. CPC C07C 17/25; B01J 6/008; B01J 19/006; B01J 2219/00763

(57) ABSTRACT

The present application relates to a system and a method for producing vinyl chloride. The system comprise a preheat unit, a gas-liquid separating unit, a heat-recovery unit, a heating unit and a thermal pyrolysis unit, and therefore heat energy of the thermal pyrolysis product can be efficiently recovered. Energy cost of the system can be efficiently lowered with the heat-recovery unit and the heating unit, and further prolonging operating cycle of the system.

### RELATED APPLICATIONS

This application claims priority to Taiwan Application Serial Number 111134647, filed on Sep. 14, 2022, which is incorporated herein by reference.

### BACKGROUND

### Field of Invention

The present application relates to a system and a method for producing vinyl chloride. More particularly, the present application provides a system and a method for producing vinyl chloride that can efficiently prolong the operating lifetime of a thermal pyrolysis furnace and reduce energy cost.

### Description of Related Art

With the development of material science, polymer materials with easy processing, light weight and excellent mechanical properties are widely used. Polyvinyl chloride is a commonly used polymer material because it has a simple production process and can be used to easily produce various types of products by general blending and molding.

Polyvinyl chloride can be formed from performing addition polymerization reaction to vinyl chloride monomer. The vinyl chloride can be obtained by subjecting 1,2-dichloroethane (ethylene dichloride, hereinafter abbreviated as EDC) to a thermal pyrolysis reaction. However, the thermal pyrolysis reaction of EDC leads energy consumption of the system to be more, and thereby a configuration able to efficiently improve heat energy recovery of the thermal pyrolysis product is necessary.

In view of this, there is an urgent need to provide a system and a method for producing the vinyl chloride to improve the defects of the conventional system and method for producing the vinyl chloride.

### SUMMARY

Therefore, an aspect of the present application is to provide a system for producing vinyl chloride. The system comprises a heat-recovery unit and a heating unit to efficiently recover and utilize heat energy of the thermal pyrolysis product, thereby lightening the loading of the heat-recovery unit, further prolonging operating lifetime of the heat-recovery unit.

Another aspect of the present application is to provide a method for producing vinyl chloride. The thermal pyrolysis reaction is performed with the aforementioned system to efficiently recover and utilize heat energy of vinyl chloride product.

According to an aspect of the present application, a system for producing vinyl chloride is provided. The system comprises a thermal pyrolysis unit, a preheat unit, a gas-liquid separating unit, a heat-recovery unit, a heating unit and a quench unit. The thermal pyrolysis unit includes a pyrolysis convection section and a pyrolysis radiation section. The thermal pyrolysis unit is configured to produce a pyrolysis gas, and the pyrolysis gas includes vinyl chloride gas, hydrochloric acid gas and non-pyrolyzed 1,2-dichloroethane gas. The preheat unit is configured to heat a raw material to obtain a preheat composition. The raw material includes 1,2-dichloroethane, and the preheat composition includes a high-temperature liquid raw material. The gas-liquid separating unit is connected between the pyrolysis convection section and the preheat unit. The gas-liquid separating unit is configured to separate a gas and a liquid, and the gas is introduced into the pyrolysis convection section through a pipeline. The heat-recovery unit is connected between the pyrolysis radiation section and the gas-liquid separating unit. The pyrolysis gas and a portion of the high-temperature liquid raw material are introduced into the heat-recovery unit to heat the portion of the high-temperature liquid raw material with the pyrolysis gas, thereby obtaining a heat-recovery composition. The heat-recovery composition comprises a first raw material vapor, and the heat-recovery composition is introduced into the gas-liquid separating unit. The heating unit is connected to the gas-liquid separating unit. A residual portion of the high-temperature liquid raw material is introduced into the heating unit to form a high-temperature composition. The high-temperature composition comprises a second raw material vapor, and the high-temperature composition is introduced into the gas-liquid separating unit. The quench unit is connected to the heat-recovery unit.

According to some embodiments of the present application, a plurality of feeding pipes are disposed at a bottom of the heat-recovery unit, and the portion of the high-temperature liquid raw material is introduced into the heat-recovery unit through the feeding pipes.

According to some embodiments of the present application, a baffle plate is disposed at one end of each of the feeding pipes.

According to some embodiments of the present application, a projection area of the baffle plate is greater than a projection area of a pipe opening of the feeding pipe.

According to some embodiments of the present application, the baffle plate is fixed at the end of each of the feeding pipes with a supporting structure, and the supporting structure protrudes from the inner wall of the heat-recovery unit.

According to some embodiments of the present application, a plurality of flow-guiding structures are disposed on a bottom surface of the baffle plate.

According to some embodiments of the present application, the heat-recovery unit includes at least one heat transfer pipe. The at least one heat transfer pipe is disposed in the heat-recovery unit, and a horizontal height of the at least one heat transfer pipe is higher than a horizontal height of each of the baffle plates.

According to some embodiments of the present application, a material countercurrent flows in the at least one heat transfer pipe relative to the high-temperature liquid raw material delivered in the feeding pipes.

According to some embodiments of the present application, a location of the gas-liquid separating unit is higher than a location of the heat-recovery unit.

According to some embodiments of the present application, an inlet pressure of the thermal pyrolysis unit is 12.1 kg/cm² G to 13.4 kg/cm² G.

According to some embodiments of the present application, an outlet pressure of the thermal pyrolysis unit is 11.0 kg/cm² G to 11.5 kg/cm² G.

According to another aspect of the present application, a method for producing vinyl chloride is provided. The method applies a thermal pyrolysis unit to produce the vinyl chloride. In the method, a heating process is firstly performed to a raw material to obtain a heating composition. The raw material includes 1,2-dichloroethane, and the heating composition includes a high-temperature liquid raw material. After the heating process is performed, a reheating process is performed to the high-temperature liquid raw material. The reheating process includes: performing a first reheating operation to a portion of the high-temperature liquid raw material to obtain a first reheating composition; performing a second reheating operation to a residual portion of the high-temperature liquid raw material to obtain a second reheating composition; and performing a gas-liquid separating process to the first reheat composition and the second reheat composition. The first reheating operation is performed to heat the portion of the high-temperature liquid raw material with a product of the thermal pyrolysis unit, and the first reheating composition comprises a first raw material vapor. The second reheating operation is performed to heat the residual portion of the high-temperature liquid raw material with a heat source, and the second reheat composition comprises a second raw material vapor. After the reheating process is performed, a thermal pyrolysis process is performed to the first raw material vapor and the second raw material vapor to form the vinyl chloride.

According to some embodiments of the present application, an inlet pressure of the thermal pyrolysis unit is 12.1 kg/cm² G to 13.4 kg/cm² G.

According to some embodiments of the present application, an outlet pressure of the thermal pyrolysis unit is 11.0 kg/cm² G to 11.5 kg/cm² G.

In the system and method for producing vinyl chloride, the heat energy of the thermal pyrolysis product can be efficiently recovered and utilized with the configuration of the heat-recovery unit, thereby reducing energy consumed by the system. Moreover, the heating unit is configured in the system to lighten the loading of the heat-recovery unit, further prolonging the operating cycle of the system. When the efficiency of the heat-recovery unit is lower at the initial stage of operating and the heat-recovery efficiency lowers at the last stage, the heating unit can efficiently provide heat energy to the high-temperature liquid raw material which does not be vaporized by the heat-recovery unit to maintain the feeding amount of pyrolysis. Besides, the baffle plate can be disposed at the feeding opening of the heat-recovery unit, thereby providing flow-guiding effect to reduce deposits at the bottom and inhibit the formation of fouling, further subjecting the interior of the heat-recovery unit to be equipped with an uniform flow field to improve heat exchange efficiency.

### BRIEF DESCRIPTION OF THE DRAWINGS

The invention can be more fully understood by reading the following detailed description of the embodiment, with reference made to the accompanying drawings as follows:

FIG. 1 illustrates a schematic configuration diagram of a system for producing vinyl chloride according to some embodiments of the present application.

FIG. 2 illustrates a schematic cross-sectional view of a heat-recovery unit according to some embodiments of the present application.

FIG. 3A illustrates an enlarged schematic cross-sectional view of an area A in FIG. 2 according to some embodiments of the present application.

FIG. 3B illustrates a schematic perspective view of a baffle plate of a feeding pipe according to some embodiments of the present application.

FIG. 3C and FIG. 3D illustrate enlarged schematic cross-sectional views of the area A in FIG. 2 according to some embodiments of the present application.

FIG. 4 illustrates a flow chart of a method for producing vinyl chloride according to some embodiments of the present application.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

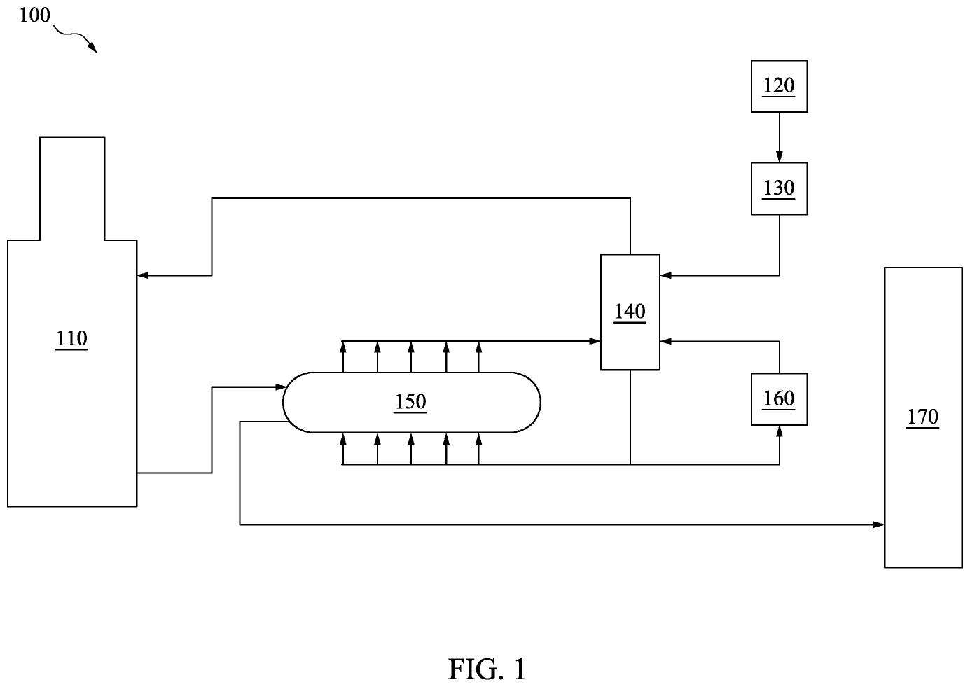

Referring to FIG. 1, which illustrates a schematic configuration diagram of a system for producing vinyl chloride according to some embodiments of the present application. The system 100 comprises a thermal pyrolysis unit 110, a raw material tank 120, a preheat unit 130, a gas-liquid separating unit 140, a heat-recovery unit 150, a heating unit 160 and a quench unit 170.

For example, the thermal pyrolysis unit 110 can be a general thermal pyrolysis furnace, and designs and configurations of the thermal pyrolysis furnace can be adjusted by one skilled in the art based on a desired thermal pyrolysis reaction. The designs and configurations of the thermal pyrolysis furnace are well known to one skilled in the art rather than focusing or mentioning them in details. The thermal pyrolysis unit 110 may have a pyrolysis convection section and a pyrolysis radiation section, and the pyrolysis radiation section is disposed under the pyrolysis convection section. An introduced gaseous reactant (1,2-dichloroethane) can be reacted into a pyrolysis gas by a thermal pyrolysis reaction of the thermal pyrolysis unit 110, and the pyrolysis gas includes vinyl chloride gas, hydrochloric acid gas and non-pyrolyzed 1,2-dichloroethane gas.

The raw material tank 120 is used to store the raw material (ethylene dichloride, hereinafter abbreviated as EDC) of the system 100. In view of stability, the EDC stored in the raw material tank 120 is liquid.

The preheat unit 130 is connected between the raw material tank 120 and the gas-liquid separating unit 140. The preheat unit 130 is used to heat the liquid EDC delivered from the raw material tank 120. There are no specific limitations to the heating method of the preheat unit 130 as long as the preheat unit 130 can apply heat energy to the EDC to increase temperature thereof. In some examples, the preheat unit 130 can heat the EDC with water vapor. Based on the adopted heat source, designs of the preheat unit 130 are well known to one skilled in the art rather than focusing or mentioning them in details. After the EDC is heated by the preheat unit 130, a preheat composition can be obtained, and the preheat composition contains high-temperature liquid EDC.

The gas-liquid separating unit 140 is connected between the preheat unit 130 and the pyrolysis convection section of the thermal pyrolysis unit 110, and a bottom of the gas-liquid separating unit 140 is connected to the heat-recovery unit 150 and the heating unit 160 through pipelines. The preheat composition heated by the preheat unit 130 is introduced into the gas-liquid separating unit 140. The high-temperature liquid EDC separated from the gas-liquid separating unit 140 is introduced into the heat-recovery unit 150 and the heating unit 160 through the bottom pipelines of the gas-liquid separating unit 140. In some embodiments, the ratio of the high-temperature liquid EDC introduced into the heat-recovery unit 150 and the heating unit 160 can be adjusted with the principle of thermosiphon. For example, the proportion of the liquid EDC introduced into the heating unit 160 can be controlled by adjusting an amount of steam in the heating unit 160 (such as, the amount of the liquid EDC introduced into the heating unit 160 is increased as an increasing of the amount of the steam). There are no specific limitations to the ratio of the high-temperature liquid EDC introduced into the heat-recovery unit 150 and the heating unit 160, and it can be adjusted by operators according to design parameters of the system 100 and reaction conditions of the thermal pyrolysis unit 110.

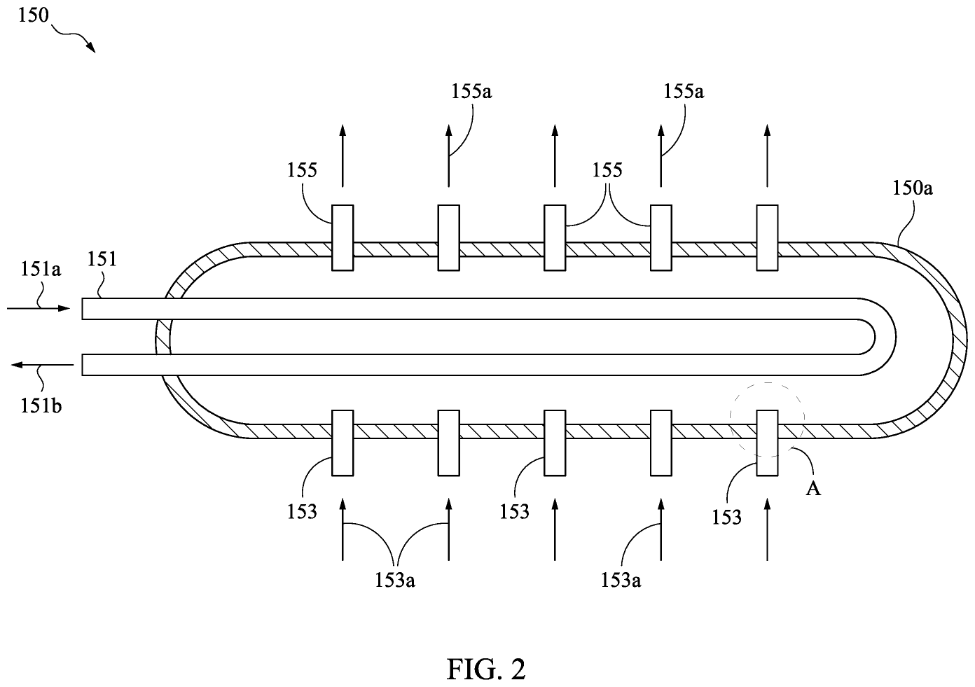

Referring to FIG. 1 together with FIG. 2, and FIG. 2 illustrates a schematic cross-sectional view of the heat-recovery unit 150 according to some embodiments of the present application. When a portion of the high-temperature liquid EDC is introduced into the heat-recovery unit 150 from the bottom of the gas-liquid separating unit 140 through the pipelines, the high-temperature liquid EDC can be introduced into an interior of a housing 150a of the heat-recovery unit 150 along a feeding direction 153a through a feeding pipe 153 at the bottom of the heat-recovery unit 150, and the high-temperature liquid EDC can be further heated to form a heat-recovery composition. The heat-recovery composition is delivered through discharge pipes 155, and further be introduced into the gas-liquid separating unit 140 through pipelines along a discharge direction 155a. In the heat-recovery unit 150, the introduced high-temperature liquid EDC can be partially phase changed into gaseous EDC vapor, and therefore the heat-recovery composition can include EDC vapor and the high-temperature liquid EDC that has not been phase changed into gaseous phase. When the heat-recovery composition is introduced into the gas-liquid separating unit 140, the EDC vapor and the high-temperature liquid EDC of the heat-recovery composition can be separated.

In the heat-recovery unit 150, heat energy is provided by the high-temperature pyrolysis gas produced by the thermal pyrolysis unit 110. The high-temperature pyrolysis gas is discharged from the pyrolysis radiation section of the thermal pyrolysis unit 110, and the high-temperature pyrolysis gas is delivered to the heat-recovery unit 150 through pipelines. The high-temperature pyrolysis gas is introduced into the housing 150a along a direction 151a through a heat transfer pipe 151, and it is discharged along a direction 151b after being circulated in the heat-recovery unit 150. Although the heat transfer pipe 151 illustrated in FIG. 2 has only one bend in the housing 150a, the present application is not limited to this configuration. The heat transfer pipe 151 can have a plurality of bends in the housing 150a for efficiently improving heat exchange efficiency of the high-temperature vinyl chloride gas. In some embodiments, as illustrated in FIG. 2, introducing position and discharging position of the high-temperature pyrolysis gas in the heat-recovery unit 150 may be disposed on the same side of the housing 150a. In other embodiments, the introducing position and the discharging position of the high-temperature pyrolysis gas in the heat-recovery unit 150 are disposed on different sides of the housing 150a based on the configurations of the heat transfer pipe 151, the configurations among the units, and the consideration of heat exchange efficiency. Because the liquid EDC is introduced from the bottom of the heat-recovery unit 150, and therefore the introducing position of the high-temperature pyrolysis gas in the heat-recovery unit 150 is higher than the discharging position of the liquid EDC (i.e. one end of the feeding pipe 153 in the interior of the heat-recovery unit 150) for achieving better heat exchange efficiency. Compared with the introduction of the liquid EDC, the configuration of the heat transfer pipe 151 is a counter-current operation to improve the heat exchange efficiency between the high-temperature pyrolysis gas and the liquid EDC.

In the heat-recovery composition obtained from the heat-recovery unit 150, there are no specific limitations to the ratio of the EDC vapor to the high-temperature liquid EDC that has not phase changed into a gas state, and it can be adjusted based on the design parameters of the system 100 and/or the heat-recovery unit 150. In some embodiments, a location of the gas-liquid separating unit 140 is higher than that of the heat-recovery unit 150. When the gas-liquid separating unit 140 is higher than the heat-recovery unit 150, the liquid EDC can be more easily introduced into the heat-recovery unit 150, and therefore the amount flowing into the heat-recovery unit 150 can be increased, further reducing an evaporation ratio of the heat-recovery unit 150 (i.e. the proportion of EDC vapor in the heat-recovery composition).

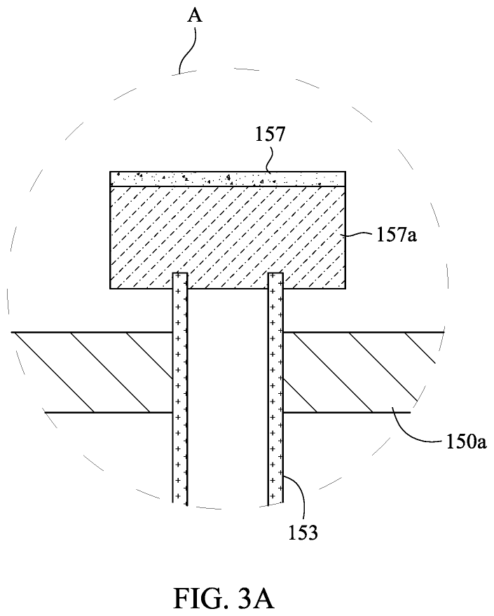

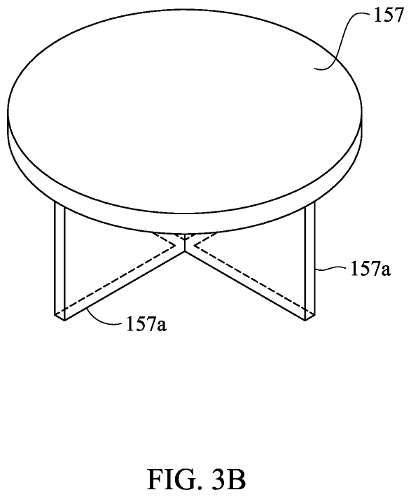

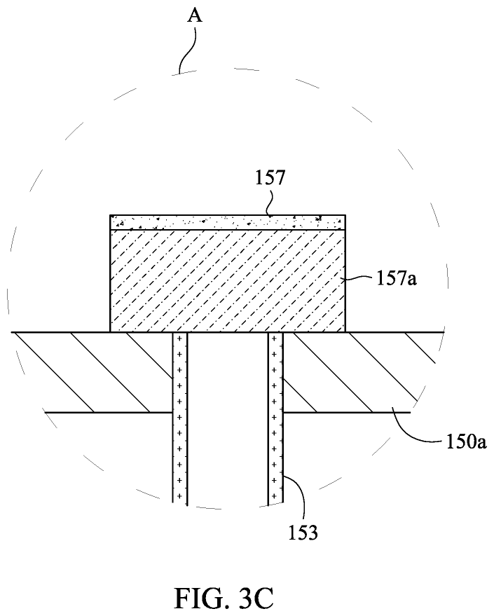

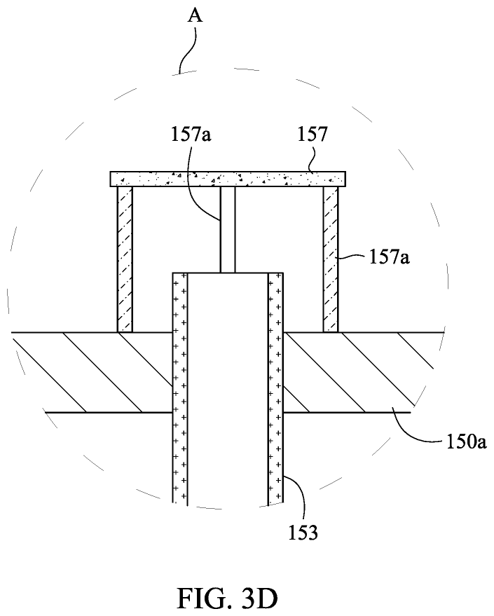

Referring to FIG. 2 together with FIG. 3A and FIG. 3B. FIG. 3A illustrates an enlarged schematic cross-sectional view of an area A in FIG. 2 according to some embodiments of the present application, and FIG. 3B illustrates a schematic perspective view of a baffle plate of a feeding pipe according to some embodiments of the present application. In the area A, a baffle plate 157 is disposed at one end of the feeding pipe 153 in the interior of the heat-recovery unit 150, and the baffle plate 157 is fixed to an opening of the feeding pipe 153 with a support 157a. As shown in FIG. 3B, the support 157a can be a cross-shaped structure disposed on a bottom surface of the baffle plate 157, and there are no specific limitations to a height of the support 157a as long as there is a suitable distance between the bottom surface of the support 157a and the opening of the feeding pipe 153 to ensure that the liquid EDC can be introduced into the heat-recovery unit 150. When the opening of the feeding pipe 153 is provided with the baffle plate 157, the introduced liquid EDC can form an appropriate flow field near the opening of the feeding pipe 153 with the flow-guiding of the baffle plate 157, thereby reducing deposits at the bottom of the heat-recovery unit 150, further inhibiting the formation of fouling, and therefore the operating lifetime of the heat-recovery unit 150 can be prolonged. Moreover, the baffle plate 157 facilitates to optimize internal flow filed of the heat-recovery unit 150, thereby improving the heat exchange efficiency. The conjugation between the baffle plate 157 and the opening of the feeding pipe 153 can be achieved by welding, clamping, locking, other suitable methods, or a combination thereof.

In some embodiments, a projection area of the baffle plate 157 is not smaller than a projection area of the opening of the feeding pipe 153. The projection area of the baffle plate 157 is larger than the projection area of the opening of the feeding pipe 153 to obtain a better flow-guiding effect. In these embodiments, the center of the baffle plate 157 is aligned with the axis of the feeding pipe 153 to further enhance the flow-guiding effect of the baffle plate 157 and subject the flow field in the housing 150a to be more uniform. In other embodiments, the baffle plate 157 is not limited to a circular plate, and it can have other configurations.

Referring to FIG. 3C, which is an enlarged schematic cross-sectional view of the area A in FIG. 2 according to some embodiments of the present application. In some embodiments, the opening of the feeding pipe 153 can be fixed to be flush with the inner wall of the housing 150a, such that the support 157a can be conjugated with the opening of the feeding pipe 153 and/or the inner wall of the housing 150a. Because the opening of the feeding pipe 153 is flushed with the inner wall of the housing 150a, the baffle plate 157 can provide a better flow-guiding effect for the bottom of the heat-recovery unit 150.

The support 157a of the baffle plate 157 is not limited to the structure illustrated in FIG. 3B, and shown as FIG. 3D, the support 157a can be columnar structures extending upward from the inner wall of the housing 150a to support and fix the baffle plate 157. In other embodiments, a bottom surface of the baffle plate 157 can be equipped with flow-guiding structures to enhance the flow-guiding effect of the baffle plate 157.

Referring to FIG. 1. The high-temperature liquid EDC discharged from the bottom of the gas-liquid separating unit 140 is partially introduced into the heat-recovery unit 150 as described above, and the residual portion is introduced into the heating unit 160. When the high-temperature liquid EDC is introduced into the heating unit 160, it can be further heated to form a high-temperature composition, then being introduced into the gas-liquid separating unit 140 once again. In some embodiments, the heating of the heating unit 160 is performed with steam and/or other high-temperature mediums, or by other heating methods. Preferably, after being treated by the heating unit 160, the high-temperature liquid EDC is partially phase changed into vapor, and therefore the high-temperature composition subjected to the treatment of the heating unit 160 includes EDC vapor and high-temperature liquid EDC. Based on the configurations of the heat-recovery unit 150 and the heating unit 160, the liquid EDC discharged from the bottom of the gas-liquid separating unit 140 can be heated by the heat-recovery unit 150 and the heating unit 160, thereby improving the reheating efficiency and efficiently prolonging operating cycle of the system 100. Besides, when the system 100 is in trial run or at the initial stage of operating, the high-temperature liquid EDC discharged from the bottom of the gas-liquid separating unit 140 can be firstly introduced into the heating unit 160 due to the lack of high-temperature pyrolysis gas produced from the thermal pyrolysis unit 110, and then the proportion of high-temperature liquid EDC introduced into the heat-recovery unit 150 is gradually increased as the operating of the system 100.

The aforementioned heat-recovery composition and high temperature composition treated by the heat-recovery unit 150 and the heating unit 160 are independently introduced into the gas-liquid separating unit 140 to separate the EDC vapor and high temperature liquid EDC in the heat-recovery composition and high temperature composition, and further the separated EDC vapor is introduced into the thermal pyrolysis unit 110 to be subjected to the thermal pyrolysis reaction. Similarly, after the operation of separating with the gas-liquid separating unit 140, the high-temperature liquid EDC in the heat-recovery composition and the high-temperature composition is further introduced into the heat-recovery unit 150 and the heating unit 160 from the bottom of the gas-liquid separating unit 140 as described above. After the thermal pyrolysis reaction, the obtained high-temperature pyrolysis gas is introduced into the heat-recovery unit 150 to heat a portion of the liquid EDC introduced into the heat-recovery unit 150 in a manner of heat exchange, thereby obtaining the EDC vapor in the heat-recovery composition. After the heat exchange in the heat-recovery unit 150, the pyrolysis gas is further introduced into the quench unit 170 and other units to form the vinyl chloride liquid.

Based on the configurations of the unit in the system 100, heat energy of the product of the thermal pyrolysis unit 110 can be efficiently recovered and utilized, and the thermal pyrolysis unit 110 can provide well thermal pyrolysis performance. In some embodiments, the delivery of the liquid EDC, the EDC vapor and the vinyl chloride gas in the system 100 can be induced by the pressure difference between the units, and therefore it is unnecessary to additionally dispose pumps and/or other units that can be used to delivery materials. In some examples, the inlet pressure of the thermal pyrolysis unit 110 of the system 100 can be 12.1 kg/cm² G to 13.4 kg/cm² G, for example. In some examples, the pressure of the high-temperature pyrolysis gas (i.e. an outlet pressure of the thermal pyrolysis unit 110) obtained from the thermal pyrolysis unit 110 can be 11.0 kg/cm² G to 11.5 kg/cm² G, and its temperature may be 470° C. to 480° C. For example, the pressure of the pyrolysis gas is reduced to 9.5 kg/cm² G, and its temperature is 290° C. after the heat exchange in the heat-recovery unit 150.

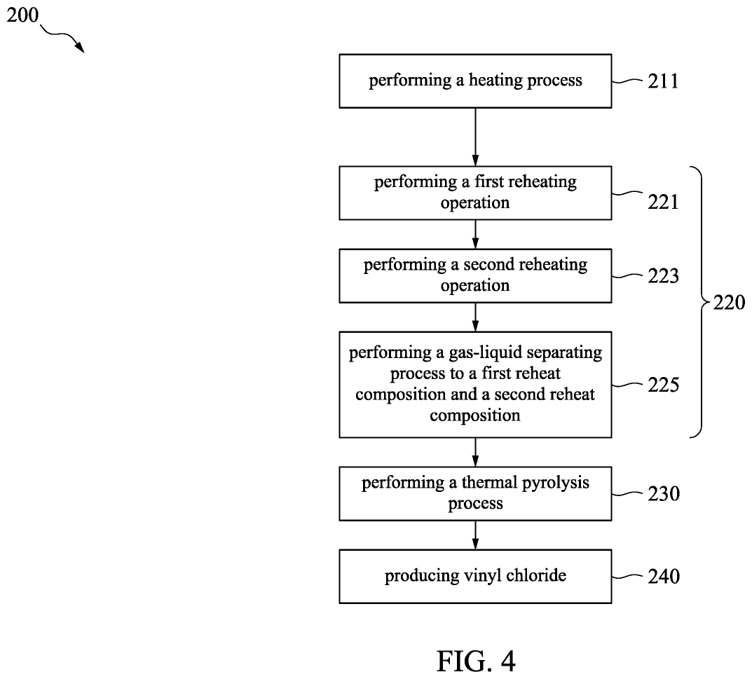

Referring to FIG. 1 together with FIG. 4. FIG. 4 illustrates a flow chart of a method 200 for producing the vinyl chloride according to some embodiments of the present application. In the method 200, a heating process is firstly performed to obtain a heating composition, shown as operation 211. The heating process utilizes the preheat unit 130 to perform a heating operation to increase temperature of the raw materials delivered from the raw material tank 120. The raw materials include 1,2-dichloroethane, and the heating composition includes a high-temperature liquid raw material. After the operation 211 is performed, the obtained heating composition is introduced into the gas-liquid separating unit 140. In the gas-liquid separating unit 140, the high-temperature liquid raw material is delivered from the bottom of the gas-liquid separating unit 140 to the heat-recovery unit 150 and the heating unit 160 to continue being subjected to a reheating process 220 because the heating composition merely includes the high-temperature liquid raw material.

During the reheating process 220, the high-temperature liquid raw material is divided into two portions. One portion is introduced into the heat-recovery unit 150 to be subjected to a first reheating operation (shown as operation 221), and the other portion is introduced into the heating unit 160 to be subjected to a second reheating operation (shown as operation 223). Because the unit bodies of the heat-recovery unit 150 and the heating unit 160 and a pipeline connected the two are all independent, it can be realized that the operation 223 can be performed before the operation 221 or the operations 221 and 223 can be simultaneously performed, in some embodiments, though FIG. 4 illustrates that the operation 221 is performed before the operation 223.

In the operation 221, the non-vaporized high-temperature liquid raw material is introduced into the heat-recovery unit 150 to be heated by the high-temperature product of the thermal pyrolysis unit 110, thereby obtaining a first reheating composition. In the heat-recovery unit 150, the higher-temperature pyrolysis gas exchanges heat with the lower-temperature (relative to the pyrolysis gas) liquid raw material, thereby subjecting a portion of the liquid raw material to be phase changed to EDC vapor, such that the first reheating composition includes the EDC vapor and the high-temperature liquid raw material. After being treated in the heat-recovery unit 150, the first reheating composition is introduced into the gas-liquid separating unit 140 to continue being subjected to the gas-liquid separation process described following.

In the operation 223, the non-vaporized high-temperature liquid raw material is introduced into the heating unit 160 to further be heated to increase temperature. During the operation 223, a portion of the liquid raw material phase changes to EDC vapor, and therefore the second reheating composition includes the EDC vapor and the high-temperature liquid raw material. In some examples, the operation 223 utilizes steam and/or other high-temperature mediums to heat the liquid raw material to obtain the second reheating composition.

When the operations 221 and 223 are performed, an amount of the high-temperature liquid raw material introduced into the heating unit 160 can be controlled based on the adjusting of amounts of the high-temperature mediums used in the operation 223, further adjusting the treating capacities of the heat-recovery unit 150 and the heating unit 160. For example, with the principle of thermosiphon, a ratio of the aforementioned treating capacities can be adjusted to meet the requirements of the method 200.

After the operations 221 and 223 are performed, the obtained first reheating composition and second reheating composition are independently introduced into the gas-liquid separating unit 140 to be subjected to a gas-liquid separating process, shown as operation 225. During the operation 225, the EDC vapor and the high-temperature liquid raw material in the first reheating composition and the second reheating composition can be separated. The separated EDC vapor can be introduced into the thermal pyrolysis unit 110 from a top of the gas-liquid separating unit 140 to be subjected to the thermal pyrolysis reaction described following, and the separated high-temperature liquid raw material can be introduced into the heat-recovery unit 150 and the heating unit 160 from the bottom of the gas-liquid separating unit 140 to subjected to the aforementioned first reheating operation and second reheating operation once again. Accordingly, the heat energy of the pyrolysis gas can be efficiently recovered by the reheating process, and further it facilitates to substantially reduce energy cost required for the method 200. Besides, the reheating process can be more flexibly performed based on the performing of the second reheating operation, and therefore, at the initial stage of the thermal pyrolysis reaction, it facilitates to utilize the heating unit 160 to solve defects of the lack of the pyrolysis gas in the heat-recovery unit 150 to efficiently enhance efficacy of the preheating process 220. It can be realized that another heating composition (i.e. a product obtained from the heating process) heated by the preheat unit 130 is also introduced into the gas-liquid separating unit 140 in addition to the first reheating composition and the second reheating composition during the operation 225. Accordingly, the gas-liquid separating unit 140 is operated to separate the EDC vapor and the high-temperature liquid raw material in the first reheating composition, the second reheating composition and the heating composition during the operation 225 because the method 200 is performed continuously.

After the reheating process 220 is performed, the obtained EDC vapor (including the EDC vapor in the aforementioned first reheating composition and second reheating composition) is introduced into the thermal pyrolysis unit 110 to be subjected to the thermal pyrolysis process to form the pyrolysis gas containing vinyl chloride gas, shown as operation 230 and operation 240. It can be realized that the high-temperature liquid raw material (comprising the high-temperature liquid raw material in the aforementioned first reheating composition and second reheating composition, and the another heating composition which has been heated by the preheat unit 130) which is separated from the gas-liquid separating unit 140 is introduced into the heat-recovery unit 150 and the heating unit 160 to continue being subjected to the aforementioned reheating process 220 after the reheating process 220 is performed. In some examples, the inlet pressure of the thermal pyrolysis process is 12.1 kg/cm² G to 13.4 kg/cm² G, and after the thermal pyrolysis reaction is performed, the pressure and temperature of the pyrolysis gas are 11.0 kg/cm² G to 11.5 kg/cm² G and 470° C. to 480° C. Based on general knowledge of the present application, the aforementioned pressure and temperature of the pyrolysis gas are respectively the outlet pressure and temperature of the thermal pyrolysis unit 110. It can be realized that the high-temperature pyrolysis gas obtained from the thermal pyrolysis process is introduced into the heat-recovery unit 150 to be subjected to the aforementioned first reheating operation, thereby efficiently utilizing the heat energy of the high-temperature pyrolysis gas.

Therefore, in the system and the method for producing the vinyl chloride of the present application, based on the configuration of the heat-recovery unit and the heating unit, the first reheating operation and the second reheating operation facilitate to efficiently recover and utilize the heat energy of the pyrolysis product and efficiently lighten the loading of the heat-recovery unit, thereby prolonging the operating lifetime of the system. Moreover, a baffle plate can be disposed at one end of the feeding pipe of the heat-recovery unit to reduce the deposits at the bottom of the heat-recovery unit, thereby efficiently inhibiting the formation of fouling, and further the flow-guiding effect of the baffle plate facilitates the interior of the heat-recovery unit to form an uniform flow field, therefore improving heat exchange efficiency.

As is understood by a person skilled in the art, the foregoing preferred embodiments of the present application are illustrated of the present application rather than limiting of the present application. In view of the foregoing, it is intended to cover various modifications and similar arrangements included within the spirit and scope of the appended claims. Therefore, the scope of which should be accorded the broadest interpretation so as to encompass all such modifications and similar structure.

What is claimed is:

1. A system for producing vinyl chloride, comprising a thermal pyrolysis unit, a preheat unit, a gas-liquid separating unit, a heat-recovery unit, a heating unit and a quench unit, wherein the thermal pyrolysis unit is configured to produce a pyrolysis gas including vinyl chloride gas, the preheat unit is configured to heat a raw material including 1,2-dichloroethane to obtain a preheat composition including a high-temperature liquid raw material, and the quench unit is connected to the heat-recovery unit;
characterized in that:
the gas-liquid separating unit is connected between a pyrolysis convection section of the thermal pyrolysis unit and the preheat unit, the heat-recovery unit is connected between a pyrolysis radiation section of the thermal pyrolysis unit and the gas-liquid separating unit, and the heating unit is connected to the gas-liquid separating unit;
wherein the gas-liquid separating unit is configured to separate a gas and a liquid, and the gas is introduced into the pyrolysis convection section through a pipeline;
wherein the pyrolysis gas and a portion of the high-temperature liquid raw material are introduced into the heat-recovery unit to heat the portion of the high-temperature liquid raw material with the pyrolysis gas, thereby obtaining a heat-recovery composition comprising a first raw material vapor, and the heat-recovery composition is introduced into the gas-liquid separating unit;
wherein a residual portion of the high-temperature liquid raw material is introduced into the heating unit to form a high-temperature composition comprising a second raw material vapor, and the high-temperature composition is introduced into the gas-liquid separating unit.

2. The system for producing the vinyl chloride of claim 1, wherein a plurality of feeding pipes are disposed at a bottom of the heat-recovery unit, and the portion of the high-temperature liquid raw material is introduced into the heat-recovery unit through the feeding pipes.

3. The system for producing the vinyl chloride of claim 2, wherein a baffle plate is disposed at one end of each of the feeding pipes.

4. The system for producing the vinyl chloride of claim 3, wherein a projection area of the baffle plate is greater than a projection area of a pipe opening of the feeding pipe.

5. The system for producing the vinyl chloride of claim 3, wherein the baffle plate is fixed at the end of each of the feeding pipes with a supporting structure, and the supporting structure protrudes from an inner wall of the heat-recovery unit.

6. The system for producing the vinyl chloride of claim 3, wherein a plurality of flow-guiding structures are disposed on a bottom surface of the baffle plate.

7. The system for producing the vinyl chloride of claim 3, wherein the heat-recovery unit includes:
at least one heat transfer pipe, disposed in the heat-recovery unit, and a horizontal height of the at least one heat transfer pipe is higher than a horizontal height of each of the baffle plates.

8. The system for producing the vinyl chloride of claim 7, wherein a material countercurrent flows in the at least one heat transfer pipe relative to the high-temperature liquid raw material delivered in the feeding pipes.

9. The system for producing the vinyl chloride of claim 1, wherein a location of the gas-liquid separating unit is higher than a location of the heat-recovery unit.

10. The system for producing the vinyl chloride of claim 1, wherein an inlet pressure of the thermal pyrolysis unit is 12.1 kg/cm² G to 13.4 kg/cm² G.

11. The system for producing the vinyl chloride of claim 1, wherein an outlet pressure of the thermal pyrolysis unit is 11.0 kg/cm² G to 11.5 kg/cm² G.

12. A method for producing vinyl chloride, wherein the method applies a thermal pyrolysis unit to produce the vinyl chloride, and the method comprises:
performing a heating process to a raw material to obtain a heating composition, wherein the raw material includes 1,2-dichloroethane, and the heating composition includes a high-temperature liquid raw material;
after the heating process is performed, performing a reheating process to the high-temperature liquid raw material, wherein the reheating process includes:
performing a first reheating operation to a portion of the high-temperature liquid raw material to obtain a first reheating composition, wherein the first reheating operation is performed to heat the portion of the high-temperature liquid raw material with a product of the thermal pyrolysis unit, and the first reheating composition comprises a first raw material vapor;
performing a second reheating operation to a residual portion of the high-temperature liquid raw material to obtain a second reheating composition, wherein the second reheating operation is performed to heat the residual portion of the high-temperature liquid raw material with a heat source, and the second reheat composition comprises a second raw material vapor; and
performing a gas-liquid separating process to the first reheat composition and the second reheat composition;
after the reheating process is performed, a thermal pyrolysis process is performed to the first raw material vapor and the second raw material vapor to form the vinyl chloride.

13. The method for producing the vinyl chloride of claim 12, wherein an inlet pressure of the thermal pyrolysis unit is 12.1 kg/cm² G to 13.4 kg/cm² G.

14. The method for producing the vinyl chloride of claim 12, wherein an outlet pressure of the thermal pyrolysis unit is 11.0 kg/cm² G to 11.5 kg/cm² G.
